(19) United States
(12) Patent Application Publication
(10) Pub. No.: US 2003/0170835 A1
(43) Pub. Date: Sep. 11, 2003

(54) Esterase, its DNA, its overexpression and production of optically active aryl propionic acids using the same

(75) Inventors: Bong Hyun Chung, Seo-ku (KR); Eun Gyo Lee, Yusung-ku (KR); Moon Sun Hahm, Yusung-ku (KR); Yeon Woo Ryu, Seoul (KR); Han Seung Lee, Yusung-ku (KR)

Correspondence Address: Finnegan, Henderson, Farabow,, Garrett & Dunner, L.L.P., 1300 I Street, N.W., Washington, DC, 20005-3315, US

(73) Assignee: KOREA RESEARCH INSTITUTE OF BIOSCIENCE AND BIOTECHNOLOGY

(21) Appl. No.: 10/118,047

(22) Filed: Apr. 9, 2002

(30) Foreign Application Priority Data
Jan. 17, 2002 (KR) 2002-2809

(51) Int. Cl. C12P 7/40; C07H 21/04; C12P 21/02; C12N 5/06; C12N 9/16

(52) U.S. Cl. 435/136; 435/69.1; 435/196; 435/320.1; 435/325; 536/23.2

(57) ABSTRACT

The present invention relates to an esterase, its DNA, its overexpression and a method for preparing an optically active aryl propionic acid of formula (1) using the same in high yield,

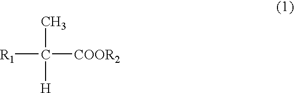

wherein R₁ represents an aryl group; and R₂ represents a hydrogen atom. □SEQ:ID□

<110>KRIBB

<120>Novel esterase, its DNA, overexpression, and production of optically active aryl propionic acids using same

<160>8

<170>Kopatentln 1.71

<210>1

<211>1143

<212>DNA

<213>Pseudomonas sp. BHY-1(KCTC 0688BP)

<400>1

atgcagattc agggacatta cgagcttcaa

| | |
|---|---|
| ttcgaagcgg tgcgcgaagc tttcgccgca | 60 |

ctgttcgacg atccccagga acgcggcgcc

| | |
|---|---|
| gcgttgtgca tccgggtcgg cggggaaacc | 120 |

gtcctcgacc tctggtccgg caccgccgac

| | |
|---|---|
| aaggacggcg ccgaggcctg gcacagcgac | 180 |

<213>Pseudomonas sp. BHY-1(KCTC 0688BP)

<400>2

```
Met Gln Ile Gln Gly His Tyr Glu Leu Gln Phe Glu
  1 5 10
Ala Val Arg Glu Ala Phe Ala Ala Leu Phe Asp Asp
            15 20
  Pro Gln Glu Arg Gly Ala Ala Leu
25 30
```

### FIELD OF THE INVENTION

The present invention relates to an esterase, its DNA, its overexpression and a method for preparing optically active aryl propionic acids using the same in high yield. More particularly, the present invention relates to an esterase having a stereoselective hydrolyase activity, its manufacturing method for mass production by employing recombiant E. coli expression system and a method for preparing optically active aryl propionic acids expressed by the following formula (1) using the same,

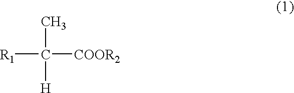

wherein R₁ represents an aryl group; and R₂ represents a hydrogen atom.

### BACKGROUND OF THE INVENTION

Indeed, FDA's (Food and Drug Administration's) Policy Statement for Development of New Drugs recommends “that the pharmacokinetic profile of each isomer should be characterized in animals and later compared to the clinical pharmacokinetic profile obtained in Phase I” drug testing. Thus, the demand for racemic switch technologies to produce each pure isomer has been rapidly increased in recent years.

Aryl propionic acids are non-steroidal anti-inflammatory drugs and known as profen drugs such as ibuprofen, ketoprofen, naproxen, flurbiprofen, fenoprofen, suprofen and the like. It is generally alleged that the (S)-profens has the higher pharmacological effect of the racemic mixture of profens bearing at least one benzene ring a-position to aliphatic carboxylic function. A method for preparing optically pure (S)-profen drugs involves the conversion of a racemic mixture of profen ester to optically active profen carboxylic acid by reacting with a stereoselective chiral enzyme.

However, it has been recently reported that (R)-enantiomers of porfens exhibit novel therapeutic effect. Particularly, U.S. Pat. No. 6,255,347 discloses that (R)-enantiomer of ibuprofen may be used as a prophylactic and herapeutic agent in the treatment of disease such as cancers, Alzheimer's and Alzheimer's related diseases. In the method for preparing (R)-enantiomer of aryl propionic acid, a racemic mixture of aryl propionic acid is treated with an esterase to produce an ester of (S)-enantiomer of aryl propionic acid and un-reacted (R)-enantiomer of aryl propionic acid is recovered.

Further, inventors of the present invention have previously identified that Pseudomonas sp. has a stereoselective hydrolase activity and its use in the preparation of (S)-profen (KR Patent Application No. 2000-02565). U.S. Pat. No. 6,201,151 discloses a process for preparing an optically active (S)-aryl propionic acid by hydrolyzing racemic thioester of aryl propionic acid in the presence of a (S)-stereoselective lipase. KR Patent Application No. 2001-0044879 discloses a process for preparing optically pure acetylmercaptoisobutylate using Pseudomonas aeruginosa as an esterase. KR Patent Application No. 1996-14399 discloses a process for preparing optically pure aryl carboxylic acid stereoselectively from a racemic mixture of α-aryl carboxylic acid using S-(-)-α-ethyl benzylamine. KR Patent Application No. 1999-0042314 discloses a process for preparing optically active carboxylic acids and esters as drugs for the treatment of hypertension using Klebsiella pneumoniae as a hydrolase. U.S. Pat. No. 5,516,690 discloses that (S)-ketoprofen can be produced in greater than 95% purity using isolated Trichosporon laibacchii.

However, it is still unsatisfactory to produce optically pure (S)— or (R)— enantiomer of aryl propionic acid using the above-mentioned enzymes and no one has reported in the literature regarding mass production of the enzyme being used therefore.

### SUMMARY OF THE INVENTION

Accordingly, an object of the present invention is to provide an esterase having excellent stereoselectivity and its DNA sequence.

Another object of the present invention is to provide a method for producing the esterase in a mass production scale by overexpression of the esterase in recombiant E. coli.

Further object of the present invention is to provide a process for preparing optically pure aryl propionic acid in high yield using the esterase,

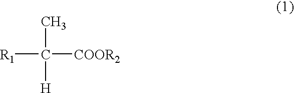

wherein R₁ represents an aryl group; and R₂ represents a hydrogen atom.

### BRIEF DESCRIPTION OF THE DRAWINGS

The above and other objects and features of the present invention will become apparent from the following description of the invention, when taken in conjunction with the accompanying drawings, in which:

FIG. 1 represents a manufacturing process of an esterase expression vector, pEESTa;

FIG. 2 represents a manufacturing process of an esterase expression vector, pEUbiESTa;

FIG. 3 represents a manufacturing process of an esterase expression vector, pErxESTa;

FIG. 4 represents an acryl amide gel electrophoresis of an esterase expression;

FIG. 5 represents an acryl amide gel electrophoresis of an esterase purified via anion exchange chromatography; and

FIG. 6 represents an acryl amide gel electrophoresis of an esterase purified via gel chromatography.

### DETAILED DESCRIPTION OF THE PREFERRED EMBODIMENTS

The present invention provides an esterase having excellent stereoselectivity, its DNA and its mass production by transformation thereof. In accordance with one aspect of the invention, there is provided a method for preparing optically pure aryl propionic acid of formula (1) using the same esterase,

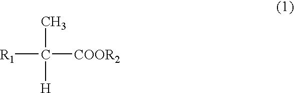

wherein R₁ represents an aryl group; and R₂ represents a hydrogen atom.

The present invention is described in detail as set forth hereunder. The esterase of the present invention is identified by the SEQ. ID. NO: 1 for its gene and the SEQ. ID. NO: 2 for its amino acid sequence and has a molecular weight of 41 kDa.

Further, the esterase derived from Pseudomonas sp. BHY-1 hydrolyzes racemic ester of a carboxylic acid unsymmetrically to produce the corresponding optically pure carboxylic acid. On the other hand, Pseudomonas sp. BHY-1 has a stereoselective hydrolase activity to convert racemic ester of aryl propionic acid to one-enantiomer aryl propionic acid. The racemic ester of aryl propionic acid used as a substrate may be prepared from racemic profen by a conventional method. Examples of the profen include ketoprofen, ibuprofen, naproxen, flurbiprofen, fenoprofen, suprofen and the like.

The inventors of the invention have selected Pseudomonas sp. BHY-1 exhibiting excellent stereoselectivity from soil and analyzed gene sequence of the esterase to obtain optically pure aryl propionic acid. Further, the present invention provides a construction of a recombiant E. coli expression vector to produce the esterase in an industrial scale. A novel esterase expression vector named as a pEESTa is constructed by introducing an NdeI restriction site to the N-terminal of the esterase and an XhoI restriction site to the C-terminal, performing PCR (Polymerase chain reaction) to amplify DNA fragments, and incorporating a T7 promoter and T7 terminator. Other pEUbiESTa and pETrxESTa vectors are also constructed by introducing ubiquitin and thioredoxin to improve production efficiency. These vectors are also incorporated with T7 promoter and T7 terminator and produce an active esterase more effectively. Theses expression vectors are then transformed into E. coli to produce E. coli transformants BL21(DE3)/pEESTta, BL21/ pEUbiESTa, and BL21/ pETrxESTa.

The obtained E. coli transformants BL21(DE3)/pEESTta is cultured and the cultured E. coli is then recovered. Examples of the profen used as the substrate to identify an activity of the obtained recombiant esterase include ethyl esters of ibuprofen, ketoprofen, naproxen, and flurbiprofen. As a result, an enantiomeric excess (eeₚ) of (S)-enantiomer profen produced by using the recombiant esterase of the present invention is not lower than 98%. It is preferable to maintain the pH in the range of from 6.0 to 12.0, more preferably from 8.0 to 10.0 and a temperature of from 15 to 80 ° C., more preferably 30 to 80° C. during resolution of aryl carboxylic acids. The obtained recombiant esterase may be purified by ion exchange chromatography, metal affinity chromatography or gel chromatography.

This invention is explained in greater detail based on the following Examples but they should not be construed as limiting the scope of this invention.

### Preparation Example

Preparation of racemic profen ethyl ester

Racemic profen (30 g) and ethanol (100 mL) were mixed and reacted in the presence of hydrosulfuric acid (2.5 mL) at 90° C. for 5 hours. The unreacted ethanol was removed by evaporation under the pressure. The reaction mixture was extracted with 1 M of sodium bicarbonate solution three times to obtain racemic profen ethyl ester.

$$\begin{matrix}Conversion ( \% ) = \frac{Conc . of ( S ) - arylpropionic acid + Conc . of ( R ) - arylpropionic acid}{Conc . of arylpropionic ester \\ \\} \times 100 & \text{Equation 1} \\ Enantiomeric excess ( \% ) = \frac{Conc . of ( S ) - arylpropionic acid - Conc . of ( R ) - arylpropionic acid}{Conc . of ( S ) - arlypropionic acid + Conc . of ( R ) - arylpropionic acid} \times 100 & \text{Equation 2}\end{matrix}$$

### Example 1

Sequence Analysis of a Novel Esterase Gene

Chromosomal DNA isolated from Pseudomonas sp. BHY-1 was partially digested with Sau3A, ligated with BamHI-digested pUC119 vector and then was transformed into E. coli DH5μ. One of the clones, carrying a plasmid named as pT7HY (about 3 kb), exhibited enzymatic activity producing (S)— ketoprofen from (R, S)-ketoprofen ester and was chosen for the further study. And also, the results showed that the novel esterase gene has a molecular weight of about 41 kDa. Transformants were selected based on a tributyrin hydrolysis as well as a stereoselectivity towards ketoprofen ester. The results showed that the novel esterase gene has a molecular weight of about 41 kDa and consists of 1,143 bp nucleotides (381 amino acids). The gene was registered in Genbank of NCBI and was assigned the Reg. No. AF380303 but has not been published yet. The novel esterase is identified by the SEQ. ID. NO: 1 for its gene and the SEQ. ID. NO: 2 for its amino acid sequence.

### Example 2

Construction of an Expression Vector for a Novel Esterase Gene

Chromosomal DNA isolated from Pseudomonas sp. BHY-1 was partially digested with Sau3A, ligated with BamHI-digested pUC119 vector and then was transformed into E. coli DH5μ. One of the clones, carrying a plasmid named as pT7HY (about 3 kb), exhibited enzymatic activity producing (S)-ketoprofen from (R, S)-ketoprofen ester. The novel estererase cDNA coding sequence was amplified by PCR using pT7HY as a template. The primers used in the above PCR are as follows.

| | |
|---|---|
| N-terminal primer | |
| 5′-GGG AAT TTC CAT ATG CAG ATT CAG GGA CAT TAC GAG CTT CAA TTC-3′ | [SEQ.ID.NO:3] |
| C-terminal primer | |
| 5′-CCG CTG GAG TTA CAG ACA AGT GGC TAG TAC CCG CGC CAG-3′ | [SEQ.ID.NO: 4] |

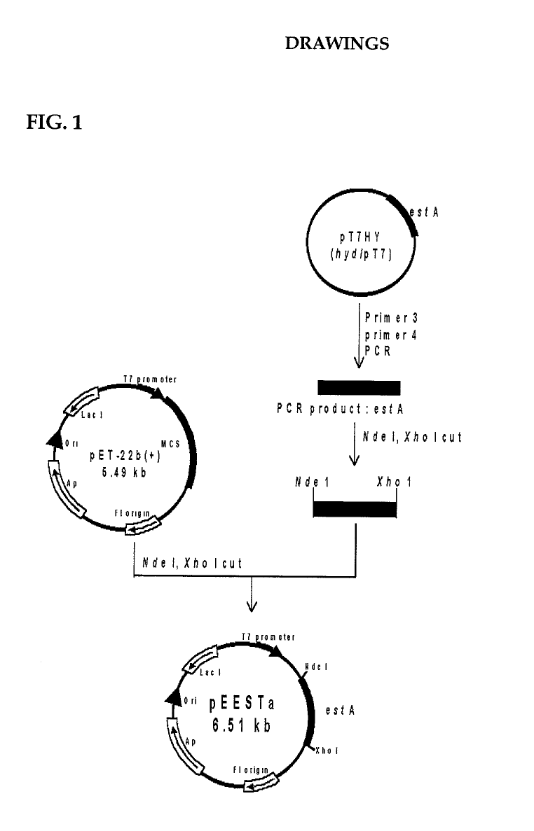

The N-terminal primer was introduced with an NdeI restriction site and also ATG was introduced as an initiation codon in place of GTG, whereas the C-terminal primer was introduced with an XhoI restriction site. The product, about 1,100 bp in size, obtained from the above PCR was double-digested with NdeI and XhoI and then separated on an agarose gel. The novel esterase gene fragment isolated from the above agarose gel was ligated into a 5,400 bp DNA fragment of pET22b(Novagen Co., Ltd., U.S.), an E. coli expression vector, double-digested with NdeI and XhoI by using a ligase. Then, an expression vector was constructed so that the novel esterase gene can be expressed, wherein its gene translation is carried out by T7 promoter and T7 terminator, and was named as pEESTa (FIG. 1). The vector pEESTa was then transformed into E. coli BL21(DE3) according to Simanis. The transformed E. coli BL21(DE3)/pEESTa was deposited to the Genebank of KRIBB on Nov. 20, 2001 and assigned the Accession No. KCTC 10122BP.

### Example 3

Expression of a Novel Esterase Gene

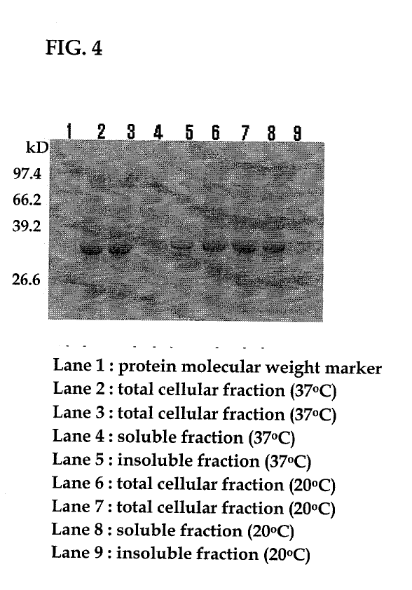

The above E. coli transformant BL21(DE3)/pEESTa(KCTC 10122BP) was cultured in a solid LB medium(yeast extract 0.5%, tryptone 1%, and NaCl 1%). The above cultured E. coli was inoculated into a liquid LB medium containing ampicillin (50 μg/mL), and then re-cultured at 37° C. until the OD₆₀₀ reached 0.6. Then, the culture was added with isopropylthio-β-D-galactoside (IPTG) to the final concentration of 1 mM and cultured further for 4 hr for the expression of an esterase gene. Cold shock response was employed for the production of an active esterase because an esterase becomes in the form of an insoluble inclusion body, which has little enzyme activity, when E. coli transformant BL21(DE3)/pEESTa(KCTC 10122BP) is produced by culturing at 37 ° C. (FIG. 4). Cold shock response is a method to produce an active enzyme wherein a given culture is incubated at 37° C. until the expression is induced by IPTG followed by lowering the culturing temperature to 5-25° C (Pamela G. Jones & Masayori Inouye, The cold-shock response, Mol. Microbiol., 11, 5,1994).

### Example 4

Construction of an Expression Vector for a Novel Ubiguitin-Fused Esterase Gene

A 228 bp fragment encoding ubiquitin (76 amino acids) was amplified by PCR using Saccharomyces cerevisiae genomic DNA as template. The primers used in the PCR are as follows.

| | |
|---|---|
| N-terminal primer | |
| 5′-GGG AAT TTC CAT ATG CAC CAC CAC CAC CAC CAC CAA ATT TTC GTC AAA ACT CTA ACA-3′ | [SEQ.ID.NO:5] |
| C-terminal primer | |
| 5′-ACC ACC CCT CAA CCT CAA GAC-3′ | [SEQ.ID.NO: 6] |

The N-terminal primer was introduced an NdeI restriction site while the C-terminal primer, where a novel esterase is to be ligated, was treated blunt-ended. The product (fragment 1: 228 bp) obtained from the above PCR was digested with NdeI and then separated on an agarose gel.

The coding region of novel esterase was isolated by PCR. The primers used in the above PCR are as follows.

| | | |
|---|---|---|
| N-terminal primer | 5′-CAG ATT CAG GGA CAT TAC GAG CTT CAA TTC-3′ | [SEQ.ID.NO:7] |
| C-terminal primer | 5′-CCC CTC GAG TTA CAG ACA AGT GGC TAG TAC CCG-3′ | [SEQ.ID.NO:4] |

The N-terminal primer was treated blunt-ended so that it can be ligated to ubiquitin sequence and then introduced with an XhoI restriction site. The product (fragment 2: 1,100 bp) obtained from the above PCR was digested with XhoI and then separated on an agarose gel.

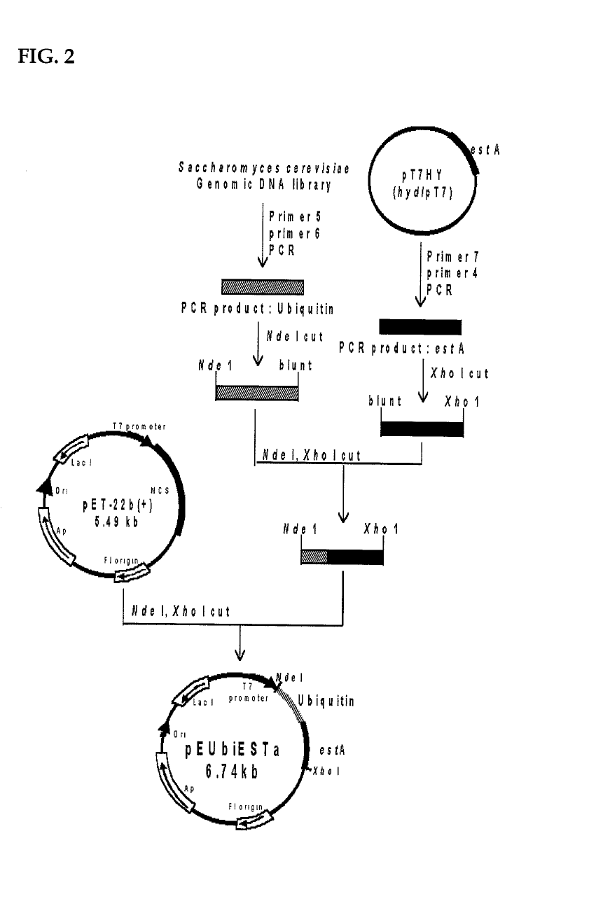

The novel esterase gene fragment as well as the ubiquitin gene fragment (PCR-amplified product) isolated from the above agarose gels were ligated into a 5,400 bp DNA fragment of pET22b(Novagen Co., Ltd., U.S.), which was digested with NdeI and XhoI by using a ligase. Then, an expression vector was constructed so that an esterase can be expressed, wherein its gene translation is carried out by T7 promoter and T7 terminator, and was named as pEUbiESTa (FIG. 2). The expression vector was not deposited because it can be readily constructed by a person with the skill in the pertinent art.

### Example 5

Construction of an Expression Vector for a Novel Thioredoxine-Fused Esterase Gene

In order to increase the rate of production and expression of the novel esterase having an activity, an expression vector introduced with thioredoxine was constructed. The novel estererase cDNA coding sequence was amplified by PCR using pT7HY as a template. The primers used in the above PCR are as follows.

| | | |
|---|---|---|
| N-terminal primer | 5′-CCG GAA TTC CAG GGA CAT TAC GAG GTT CAA TTC-3′ | [SEQ.ID.NO:8] |
| C-terminal primer | 5′-CCG CTC GAG TTA CAG ACA AGT GGC TAG TAC CCG-3′ | [SEQ.ID.NO:4] |

N-terminal of primers were treated with EcoRI so that they can be ligated to thioredoxine sequences and then introduced with an XhoI restriction site. The PCR product (1,100 bp) was gel purified and digested with EcoRI and Xhol.

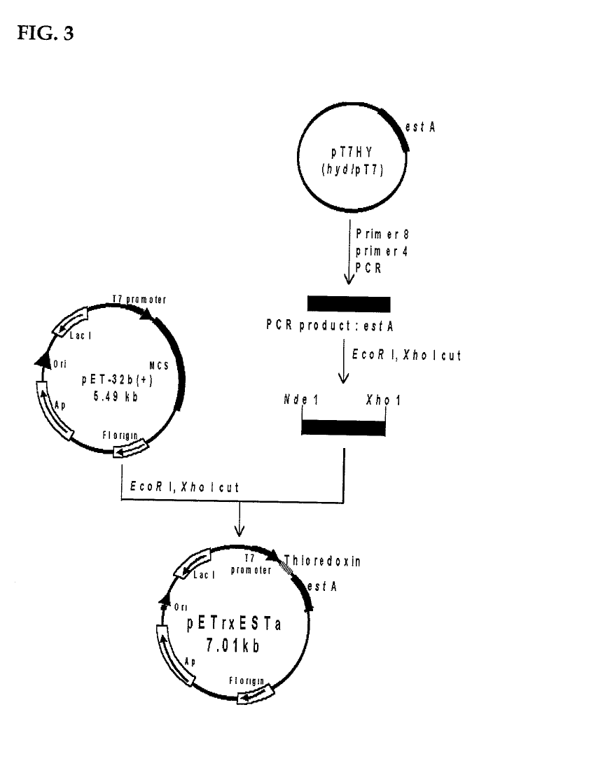

The novel esterase gene fragment isolated from the above agarose gel was ligated into a 5,900 bp DNA fragment of pET32b(Novagen Co., Ltd., U.S.), an E. coil expression vector that contains thioredoxine which was double-digested with EcoRI and XhoI, by using a ligase. Then, an expression vector was constructed so that the esterase can be expressed, wherein its gene translation is carried out by T7 promoter and T7 terminator, and was named as pETrxESTa (FIG. 3). The expression vector was not deposited because it can be readily constructed by a person with the skill in the pertinent art. The two fusion partners have six histidine tags and are thus easily purified and are also characterized in that they have special cleavage sites for ubiquitin hydrolase and enterokinase (FIGS. 2 and 3).

### Example 6

Expression of an Esterase

The above E. coli transformant BL21(DE3)/pEESTa(KCTC 10122BP) was inoculated into an LB medium and cultured at 37 ° C. until the OD₆₀₀ reached 0.6. Then, the culture was added with IPTG to the final concentration of 1 mM and cultured further for 4 hr to induce the expression of the fused esterase gene. The expressed fused esterase was identified on an SDS-PAGE gel (12% acrylamide) (see Example 3) and compared with the esterase in the Example 3(FIG. 4). Cold shock response was employed for the production of an active esterase because an esterase becomes in the form of an insoluble inclusion body, which has little enzymatic activity, when E. coli transformant BL21(DE3)/pEESTa(KCTC 10122BP) is produced by culturing at 37° C. (FIG. 4). It is noteworthy that the culture is incubated at 37 ° C. until the expression is induced by IPTG followed by lowering the culturing temperature to 20° C., whereby the esterase is produced in an active form. The result showed that the above two fused proteins of ubiquitin-esterase and thioredoxine-esterase, which were both produced by cold shock response, were shown to retain their optical selectivity and hydration capability.

### Example 7

Identification of a Novel Esterase Expression

The culture was centrifuged for 20 min at 7,000 rpm and the cells were recovered. To study the expression level of the esterase that is expressed, the whole cell was divided into a soluble fraction and an insoluble fraction via sonication and its expression was examined. Three samples such as a whole fraction, a soluble fraction and an insoluble fraction, was dissolved in 100 μL of protein solubilizing buffer solution(12 mM Tris-HCl pH 6.8, 5% glycerol, 2.88 mM mercaptoethanol, 0.4% SDS, 0.02% bromophenol blue) and then heated for 5 min at 100° C. Ten μL each of thus formed solutions was loaded onto a polyacrylamide gel, wherein a 0.75 mm thick 12% gradient separating gel (pH 8.8, 20 cm(W)×10 cm(H)) was covered with a 5% stacking gel (pH 6.8, 10 cm(W)×12 cm(H)). Then, electrophoresis was performed for 80 min (120 V, 60 mA) and the gel was stained with Coomassie Blue. The gel scanning (BioRad, Imaging Densitometer GS-700, U.S.) result of the esterase revealed that the expression level after IPTG induction was 46.7%, and 94.2% of the total expression was present in the form of an insoluble inclusion body.

### Example 8

Purification of a Novel Esterase via Anion Exchange Chromatography

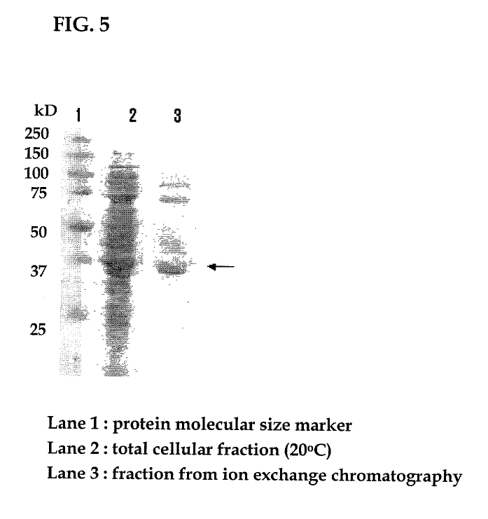

Ion exchange chromatography was performed to purify the novel esterase produced from the recombinant E. coli. The chromatography was performed by using Q-Sepharose(Pharmacia Co., Ltd., Sweden) as a resin at pH 8.5 at the rate of 4.0 mL/min. Samples were prepared by crushing cell walls of E. coil by using a sonicator followed by filtering thus obtained soluble fraction through micro filter(0.2 μm). Q-Sepharose was equilibrated with 50 mM Tris-HCl(pH 8.5) buffer solution. The esterase was fractioned by using NaCl linear gradient of an eluent buffer solution (1N NaCl/50 mM Tris-HCl, pH 8.5) wherein the sample was first put into the chromatography column followed by a thorough rinse with an equilibrium buffer solution. Thus purified esterase was identified on an SDS-PAGE gel electrophoresis as in the Example 6 (FIG. 5).

### Example 9

Purification of a Novel Esterase via Gel Chromatography

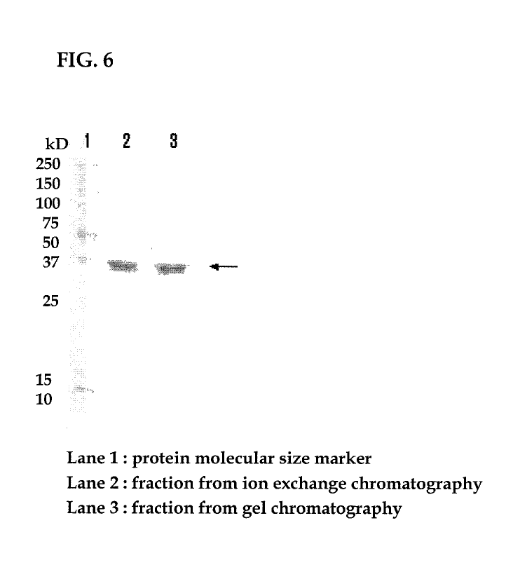

Gel chromatography was performed by using the fraction obtained from the above anion exchange chromatography. The chromatography was performed by using Sephacry S-200-HR(Pharmacia Co., Ltd., Sweden) as a resin at pH 8.5 at the rate of 0.3 mL/min. Samples were prepared by filtering the fraction obtained from the ion exchange chromatography through micro filter(0.2 μm). Sephacry S-200-HR was equilibriated with 50 mM Tris-Cl/10 mM NaCl buffer solution. The esterase was fractioned after putting the sample into the chromatography column and flowing it at the rate of 0.3 mL/min. Thus purified esterase was identified on an SDS-PAGE gel as in the Example 6(FIG. 6).

### Example 10

Effect of Optical Resolution Conditions on Optical Resolution of a Novel Esterase

1. Effect of pH

The hydration by a novel esterase is mostly performed in a buffered solution and thus the structure of the enzyme can be influenced much by the pH and chemical properties of a buffer solution being used. When using Pseudomonas sp. BHY-1 as a whole cell enzyme, the optimal enzyme activity was observed at pH 8.5. In the case of the novel esterase, the enzyme activity was shown to have a relatively wide pH range of 7-11 and the optical selectivity was shown to be optimal at pH 10.0 as shown in the following Table 1.

TABLE 1
Optimal pH of Esterase

| | | | | | | |
|---|---|---|---|---|---|---|
| PH | 7.0 | 8.0 | 8.5 | 9.0 | 10.0 | 11.0 |
| Conversion (%) | 1.8 | 2.4 | 2.0 | 2.2 | 7.9 | 5.4 |
| Enantiomeric excess (%) | 100 | 100 | 100 | 100 | 100 | 41 |

2. Effect of Temperature

Optimal temperature for optical resolution is affected by the fictive temperature, defined as racemic temperature, and the optical selectivity in response to a temperature increase tends to vary depending on the kind of an enzyme. The novel esterase of the present invention is shown to have an excellent optical selectivity and the following shows the reaction rate of the enzyme. The reaction rate was observed at 10° C.-90° C., a temperature range for culturing Pseudomonas sp. BHY-1, and the optimal reaction rate was observed at 60° C.

TABLE 2
Optimal temperature of Esterase

| | | | | | | | |
|---|---|---|---|---|---|---|---|
| Temperature (° C.) | 30 | 40 | 50 | 60 | 70 | 80 | 90 |
| Conversion (%) | 7.9 | 8.9 | 11.5 | 13.1 | 12.8 | 10.9 | 9.9 |
| Enantiomeric | 100 | 100 | 100 | 100 | 100 | 41 | 41 |
| excess (%) | | | | | | | |

3. Type of Reaction Substrates

Reaction substrates are in the form of ester and are mostly water insoluble. Therefore, it becomes necessary to mediate the reaction substrate to bind the enzyme for a desired enzyme reaction. In general, organic solvents such as dimethylsulfoxide, dimethylformamide, tetrahydrofuran, cyclohexane, benzene, etc., or non-ionic surfactant are used to serve the above mediation purpose. It is important to determine an organic solvent or a surfactant suitable for a given substrate. In profen pharmaceuticals, for example, Triton X-100 and dimethylsulfoxide were shown most effective.

### Example 11

Optical Resolution of aryl propionic acid by Using a Novel Recombinant Esterase

Hydration was performed using 20 mM esters of ibuprofen, ketoprofen, and flurbiprofen to produce optically active ibuprofen, ketoprofen, and flurbiprofen. The reaction was performed at 37° C. (pH 8.5) with a reaction volume of 500 μL. Twenty four hours after the enzyme reaction, there was about 40% of conversion and enantiomeric excess (eeₚ) was higher than 98.5% of optical selectivity as shown in the following Table 3.

TABLE 3

| Substrate | Conversion (%) | Enantiomeric excess (%) |
|---|---|---|
| Ibuprofen | 40.9 | >99 |
| Ketoprofen | 39.3 | >99 |
| Flurbiprofen | 41.4 | 99 |

The novel esterase of the present invention derived from Pseudomonas sp. BHY-1 can be used in producing optically pure (S)— or (R)— type of aryl propionic acid having a pharmaceutical activity with high efficiency from racemic aryl propionic acid.

What is claimed is:

1. An esterase gene identified by SEQ. ID. NO: 1.

2. An esterase identified by SEQ. ID. NO: 2 which has an stereoselective hydrolase activity toward racemic aryl propionic ester.

3. An expression vector containing the esterase gene in claim 1.

4. The expression vector according to claim 3, wherein said expression vector is pEESTa, pEUbiESTa or pETrxESTa.

5. A transformant transformed via said expression vector in claim 3.

6. The transromant according to claim 5, wherein said transromant is E. coli BL21(DE3)/pEESTta, BL21/ pEUbiESTa, or BL21/ pETrxESTa.

7. A method for preparing an esterase for mass production by isolation and purification of expressed esterase from the transformant in claim 5.

8. A method for preparing an optically pure (S)- or (R)-enantiomer aryl propionic acid from racemic aryl propionic acid or ester by employing said esterase in claim 2 in high yield.
